(19) United States
(12) Patent Application Publication
(10) Pub. No.: US 2026/0284847 A1
(43) Pub. Date: Sep. 24, 2026

(54) Plier

(71) Applicants: Tomasz Cychowski, Winnipeg (CA); Anna Cychowski, Winnipeg (CA); Krzystof Jerzy Cychowski, Winnipeg (CA)

(72) Inventors: Tomasz Cychowski, Winnipeg (CA); Anna Cychowski, Winnipeg (CA); Krzystof Jerzy Cychowski, Winnipeg (CA)

(21) Appl. No.: 19/085,590

(22) Filed: Mar. 20, 2025

(51) Int. Cl. B25B 27/02; A61M 39/10

(52) U.S. Cl. CPC B25B 27/02; A61M 39/10; A61M 2209/04

(57) ABSTRACT

A plier to facilitate a disengagement of a catheter and a tube includes a first elongated rod structure. The elongated rod structure includes a first jaw portion with a first engagement structure to engage with one of the catheter and the tube. The plier further includes a second elongated rod structure pivotally coupled to the first elongated rod structure and adapted to pivot between a first position and a second position. The second elongated rod structure includes a second jaw portion having a second engagement structure to engage with other of the catheter and the tube. The first elongated rod structure and the second elongated rod structure are pivoted to the second position from the first position to move the first engagement structure and the second engagement structure away from each other to disengage the catheter from the tube.

### BACKGROUND OF THE INVENTION

### 1. Technical Field

The present disclosure relates, generally, to a plier, and more particularly relates to a plier suitable for disengaging a catheter from a tube.

### 2. Description of the Prior Art

Catheters are generally connected to tubes of collecting bags via conical friction joint to assure positive seal therebetween. However, the friction created between female and male part presents difficulties when it is necessary to disconnect the tube and the catheter. Currently, tube and the catheter are manually disengaged by applying force of fingers directly to the connected components. This needs to be done a minimum of twice a day and a patient can have multiple catheters inserted, depending on the medical conditions. Some locations of the catheters present difficult conditions for the disengagement to be performed by the patient alone.

### SUMMARY OF THE INVENTION

In one aspect of the disclosure, a plier is disclosed. The plier facilitates a disengagement of a catheter and a tube, and includes a first elongated rod structure having a first jaw portion with a first engagement structure to engage with one of the catheter and the tube. The plier also includes a second elongated rod structure pivotally coupled to the first elongated rod structure and adapted to pivot between a first position and a second position. The second elongated rod structure includes a second jaw portion having a second engagement structure to engage with other of the catheter and the tube. The first elongated rod structure and the second elongated rod structure are pivoted to the second position from the first position to move the first engagement structure and the second engagement structure away from each other to disengage the catheter from the tube.

In some additional, alternative, or selectively cumulative embodiments, each of the first and second engagement structures includes a U-shaped groove to receive the catheter and the tube.

In some additional, alternative, or selectively cumulative embodiments, each of the first and second elongated rod structures includes a handle portion adapted to be manipulated by a user to pivot the second elongated rod structure relative to the first elongated rod structure.

In some additional, alternative, or selectively cumulative embodiments, the handle portions are arranged on a first side of a pivot joint of the plier and the jaw portions are arranged on a second side of the pivot joint.

In some additional, alternative, or selectively cumulative embodiments, the plier further comprises a spring attached to the first and second elongated rod structures to bias the second elongated rod structure and the first elongated rod structure to the first position.

### BRIEF DESCRIPTION OF FIGURES

FIG. 1 illustrates a top perspective view of a plier with elongated rod structures arranged in a closed position, in accordance with an embodiment of the disclosure;

FIG. 2 illustrates a top perspective view of the plier of FIG. 1 with the elongated rod structures arranged in an open position, in accordance with an embodiment of the disclosure; and

FIG. 3 illustrates the plier of FIG. 1 engaged with an assembly of catheter and a tube to disengage the catheter from the tube, in accordance with an embodiment of the disclosure.

### DETAILED DESCRIPTION

Example embodiments are described below with reference to the accompanying drawings. Unless otherwise expressly stated in the drawings, the sizes, positions, etc., of components, features, elements, etc., as well as any distances therebetween, are not necessarily to scale, and may be disproportionate and/or exaggerated for clarity.

The terminology used herein is for the purpose of describing particular example embodiments only and is not intended to be limiting. As used herein, the singular forms “a,” “an” and “the” are intended to include the plural forms as well, unless the context clearly indicates otherwise. It should be recognized that the terms “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise specified, a range of values, when recited, includes both the upper and lower limits of the range, as well as any sub-ranges therebetween. Unless indicated otherwise, terms such as “first,” “second,” etc., are only used to distinguish one element from another. For example, one element could be termed a “first element” and similarly, another element could be termed a “second element,” or vice versa. The section headings used herein are for organizational purposes only and are not to be construed as limiting the subject matter described.

Unless indicated otherwise, the terms “about,” “thereabout,” “substantially,” etc. mean that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art.

Spatially relative terms, such as “right,” left,” “below,” “beneath,” “lower,” “above,” and “upper,” and the like, may be used herein for ease of description to describe one element's or feature's relationship to another element or feature, as illustrated in the drawings. It should be recognized that the spatially relative terms are intended to encompass different orientations in addition to the orientation depicted in the figures. For example, if an object in the figures is turned over, elements described as “below” or “beneath” other elements or features would then be oriented “above” the other elements or features. Thus, the term “below” can, for example, encompass both an orientation of above and below. An object may be otherwise oriented (e.g., rotated 90 degrees or at other orientations) and the spatially relative descriptors used herein may be interpreted accordingly.

Unless clearly indicated otherwise, all connections and all operative connections may be direct or indirect. Similarly, unless clearly indicated otherwise, all connections and all operative connections may be rigid or non-rigid.

Like numbers refer to like elements throughout. Thus, the same or similar numbers may be described with reference to other drawings even if they are neither mentioned nor described in the corresponding drawing. Also, even elements that are not denoted by reference numbers may be described with reference to other drawings.

Many different forms and embodiments are possible without deviating from the spirit and teachings of this disclosure and so this disclosure should not be construed as limited to the example embodiments set forth herein. Rather, these example embodiments are provided so that this disclosure will be thorough and complete, and will convey the scope of the disclosure to those skilled in the art.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments.

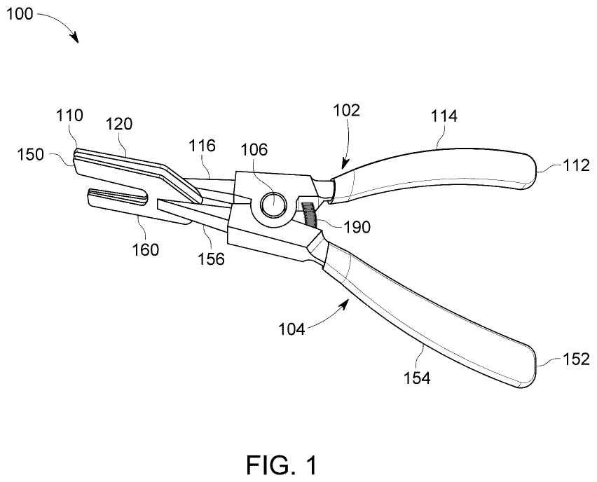

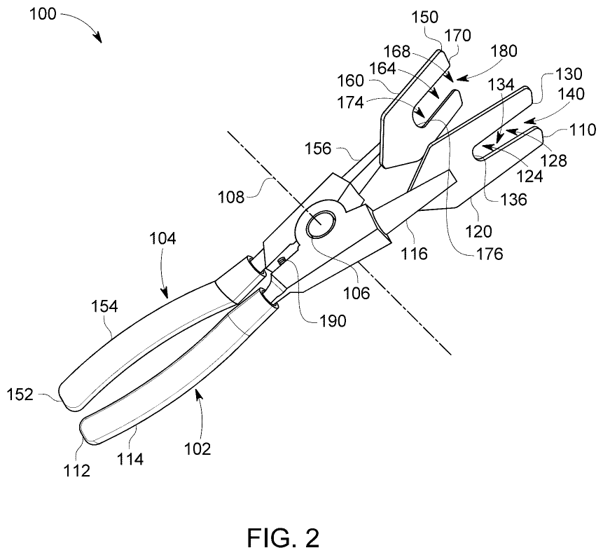

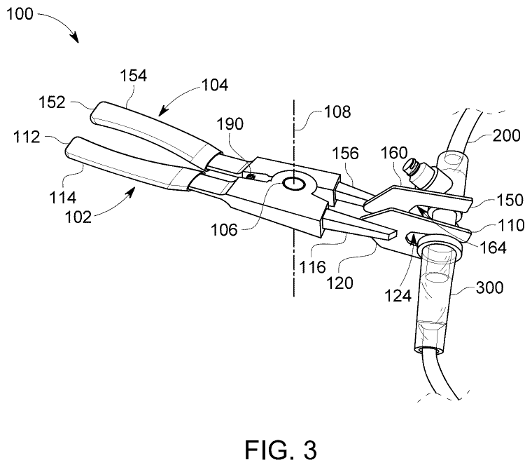

Referring to FIGS. 1 to 3, a plier 100 is shown according to an example embodiment of the disclosure. The plier 100 is suitable for disengaging a catheter 200 from a tube 300, shown in FIG. 3, and includes a pair of elongated rod structures 102, 104 pivotally attached to each other at a pivot joint 106. The elongated rod structures 102, 104 are adapted to pivot between a closed position i.e., a first position, shown in FIG. 1, and an open position i.e., a second position, shown in FIGS. 2 and 3, about a pivot axis 108. As shown, a first rod structure 102 of the pair of rod structures 102, 104 includes a first end 110, a second end 112, a handle portion i.e., a first handle portion 114 arranged on a first side of the pivot joint 108 i.e., fulcrum, and a jaw portion i.e., a first jaw portion 116 arranged on a second side of the pivot joint 108.

As shown, the first handle portion 114 extends from the second end 112 to the pivot joint 108 and provides a grip for a user to hold and manipulate the plier 100, while the first jaw portion 116 extends from the pivot joint 108 to the first end 110. In the embodiment, the first jaw portion 116 includes a first engagement structure 120 adapted to engage with the catheter 200 or the tube 300 to enable the disengagement of the catheter 200 from the tube 300.

As shown in FIGS. 1 to 3, the first engagement structure 120 includes a U shape defining a U-shaped groove 124 i.e., a first groove 124, to receive the catheter 200 or the tube 300. As shown, the first groove 124 includes a straight groove portion 128 having a first end 130 arranged at the first end 110 of the first rod structure 102, and a curved groove portion 134 arranged at a second end 136 of the straight groove portion 128. An opening 140 of the first groove 124 is defined at the first end 130 of the straight groove portion 128 to enable an insertion of the catheter 200 or the tube 300 inside the first groove 124.

Similar to the first rod structure 102, a second rod structure 104 of the pair of rod structures 102, 104 includes a first end 150, a second end 152, a handle portion i.e., a second handle portion 154, arranged on a first side of a pivot joint 106 i.e., fulcrum, and a jaw portion i.e., a second jaw portion 156, arranged on a second side of the pivot joint 106. As shown, the second handle portion 154 extends from the second end 152 to the pivot joint 106, and provides a grip for a user to hold and manipulate the plier 100. The second jaw portion 156 extends from the pivot joint 106 to the first end 150 of the second rod structure 104. In the embodiment, the second jaw portion 156 includes a second engagement structure 160 adapted to engage with the catheter 200 or the tube 300 to enable the disengagement of the catheter 200 from the tube 300.

As shown in FIGS. 1 to 3, the second engagement structure 160 includes a U shape defining a U-shaped groove 1 i.e., a second groove 164, to receive the catheter 200 or the tube 300. As shown, the second groove 164 includes a straight groove portion 168 having a first end 170 arranged at the first end 150 of the second rod structure 104, and a curved groove portion 174 arranged at a second end 176 of the straight groove portion 168. An opening 180 of the second groove 164 is defined at the first end 170 of the straight groove portion 168 to enable an insertion of the catheter 200 or the tube 300 inside the second groove 164. Moreover, in some embodiments, the plier 100 includes a spring 190 configured to bias the rod structures 102, 104 in the closed position. Accordingly, the user needs to apply a force against the spring to move the rod structures in the open position. In the open position, the handle portions 114, 154 are arranged relatively proximate to each other than the closed portion, while the jaw portions 116, 156 are disposed relatively away from each other than the closed position.

For disengaging the catheter 200 and the tube 300, a user engages the first engagement structure 120 with one of the tube 300 and the catheter 200, for example, tube 300, and the second engagement structure 160 with other of the tube 300 and the catheter 200, for example, the catheter 200. It may be noted that the first engagement structure 120 is engaged with the tube 300 by inserting a portion of the tube 300 inside the first groove 124, as shown in FIG. 3, while the second engagement structure 160 is engaged with the catheter 200 by inserting a portion of the catheter 200 inside the second groove 164. Also, the engagement structures 120, 160 are engaged with the catheter 200 and the tube 300 with the rod structures 102, 104 arranged at the closed position. Upon engaging the engagement structures 120, 160 with the tube 300 and the catheter 200, the user applies a force, against the biasing force of the spring 190, on the rod structures 102, 104 to move the rod structures 102, 104 to the open position to move the jaw portions 116, 156 i.e., engagement structures 120, 160 away from each other to disengage the catheter 200 and the tube 300. As the jaw portions 116, 156 moves away from each other, the engagement structures 120, 160 pushes the tube 300 and the catheter 200 away from each other, thereby disengaging the catheter 200 from the tube 300. In this manner, the plier 100 facilitates the user to easily disengage the tube 300 and the catheter 200 conveniently and quickly.

It should be understood that the foregoing description is only illustrative of the aspects of the disclosed embodiments. Various alternatives and modifications can be devised by those skilled in the art without departing from the aspects of the disclosed embodiments.

1. A plier to facilitate a disengagement of a catheter and a tube, the plier comprising:
a first elongated rod structure including a first jaw portion with a first engagement structure to engage with one of the catheter and the tube; and
a second elongated rod structure pivotally coupled to the first elongated rod structure and adapted to pivot between a first position and a second position, the second elongated rod structure includes a second jaw portion having a second engagement structure to engage with other of the catheter and the tube,
wherein the first elongated rod structure and the second elongated rod structure are pivoted to the second position from the first position to move the first engagement structure and the second engagement structure away from each other to disengage the catheter from the tube.

2. The plier of claim 1, wherein each of the first and second engagement structures includes a U-shaped groove to receive the catheter and the tube.

3. The plier of claim 1, wherein each of the first and second elongated rod structures includes a handle portion adapted to be manipulated by a user to pivot the second elongated rod structure relative to the first elongated rod structure.

4. The plier of claim 3, wherein the handle portions are arranged on a first side of a pivot joint of the plier and the jaw portions are arranged on a second side of the pivot joint.

5. The plier of claim 1 further comprising a spring attached to the first and second elongated rod structures to bias the second elongated rod structure and the first elongated rod structure to the first position.
